(19) United States
(12) Patent Application Publication
(10) Pub. No.: US 2021/0030256 A1
(43) Pub. Date: Feb. 4, 2021

(54) Sanitizing device

(71) Applicant: APPENNINO DI ORI VITTORIO & C. S.N.C., Argelato, BOLOGNA (IT)

(72) Inventor: Vittorio ORI, Argelato, BOLOGNA (IT)

(21) Appl. No.: 16/967,026

(22) PCT Filed: Jan. 11, 2019
(86) PCT No.: PCT/IB2019/050222

§ 371 Date: Aug. 3, 2020

(30) Foreign Application Priority Data
Feb. 6, 2018 (IT) 102018000002445

(51) Int. Cl. A47L 23/22; A61L 2/22; A61L 2/24; A61L 2/26

(52) U.S. Cl. CPC A47L 23/22; A61L 2/22; A61L 2/24; A61L 2202/11; A61L 2202/14; A61L 2202/20; A61L 2/26

(57) ABSTRACT

A sanitizing device includes a treadable mat wherein at least one sanitizing portion forms a support surface which may be walked on by a user and suitable for dispensing a sanitizing substance in the form of a vaporized jet, at least one dispensing group housed in a dispensing compartment situated below the support surface, a vaporization group having at least one tank containing the sanitizing substance in liquid state, and a vaporization unit equipped with a vaporization device to cause the sanitizing substance to instantaneously pass from liquid state to gaseous state in a vaporization chamber.

### CROSS-REFERENCE TO RELATED APPLICATIONS

This application is a National Phase Application of PCT International Application No. PCT/IB2019/050222, having an International Filing Date of Jan. 11, 2019 which claims priority to Italian Application No. 102018000002445 filed Feb. 6, 2018, each of which is hereby incorporated by reference in its entirety.

### TECHNICAL FIELD

The present disclosure concerns a sanitizing device in the form of a treadable mat, in particular a doormat, having the function of sanitizing the support surfaces of a user on the mat, in particular the soles of the shoes.

### BACKGROUND

In a previous patent application no. WO 2016/147087 A1 by the same Applicant, a mat is described with a sanitizing function.

In one embodiment, two cylinders containing a pressurized gaseous sanitizing substance are housed in the mat below the support surface of the user. In the presence of the user, the cylinders dispense the substance in the form of a spray. However, the cylinders occupy a fairly large volume, which affects the height of the mat in particular. In addition, the cylinders run out rather quickly and often need to be replaced or refilled.

In a variant embodiment, the sanitizing substance is dispensed in the form of a vaporized or nebulized jet. Under the support surface of the user there is a flattened tank suitable to contain a sanitizing liquid to be vaporized or nebulized. The tank is equipped with one or more dispensing nozzles suitable to allow the release of the sanitizing substance in the form of a vaporized or nebulized jet. The sanitizing liquid is vaporized by one or more heating cells applied to the bottom of the tank. Alternatively, the sanitizing liquid is nebulized by means of ultrasonic actuators. In both cases, however, a significant amount of time is required before the sanitizing substance changes from a liquid to a gaseous state and reaches the pressure required to be dispensed by the nozzles provided on the tank, which in any case is perceived negatively by the user.

In addition, in these embodiments, the presence and position of the dispensing nozzles depend on the position of the underlying cylinders or tanks, making the construction inflexible and limiting the possible applications of the device.

### BRIEF SUMMARY

The purpose of the present disclosure is to propose a treadable mat shaped sanitizing device that does not suffer from the limitations mentioned above with reference to the embodiments of the mat according to the prior art.

In particular, the purpose of the disclosure is to propose a mat device that allows an instantaneous delivery of the sanitizing substance and that is at the same time small in size, reliable over time and long-lasting.

### BRIEF DESCRIPTION OF THE DRAWINGS

The features and advantages of the sanitizing device according to the disclosure will become evident from the description provided hereinafter of the preferred embodiments thereof, provided by way of indicative and non-limiting examples, with reference to the accompanying figures, wherein:

FIG. 1 is a perspective view of a sanitizing device according to the disclosure, in a first embodiment; provided with two sanitizing portions;

FIG. 2 is a perspective view of the separate parts of the sanitizing device in FIG. 1;

FIG. 3 shows the components of the vaporization group and two vaporized sanitizing substance dispensing groups;

FIG. 4 is a prospective view, in axial section, of a tank of liquid sanitizing substance and a vaporization unit connected to the tank;

FIG. 4a is a perspective view, in axial section, of the vaporization unit of FIG. 4;

FIG. 5 is a perspective view, in axial section, of a vaporization unit in a variant embodiment;

FIG. 6 is a perspective view of the sanitizing device in a second embodiment;

FIG. 7 is a perspective view of the separate parts of the sanitizing device in FIG. 6;

FIG. 8 is a perspective view of the sanitizing device in a third embodiment; and

FIG. 8a is a cross-section of the sanitizing device in FIG. 8 corresponding to one of the sanitizing compartments.

### DETAILED DESCRIPTION

In the following description, the elements common to the various embodiments are indicated at the same numeric references.

In the drawings, a sanitizing device according to the disclosure has been indicated collectively at 1; 100; 1000.

In a general embodiment, the sanitizing device 1; 100; 1000, comprises a treadable mat 10; 110; 1010, for example in the form of a doormat, wherein at least one sanitizing portion 12; 1020 is obtained; suitable for sanitizing the soles or, in a particular embodiment, also the sides and upper part of a shoe, by dispensing a liquid sanitizing substance in the form of a vaporized jet.

Each sanitizing portion 12; 1020 forms a support surface 12′; 1020′ that may be walked on by a user. For example, such support surface is in the form of a grid, so as to protect the underlying devices and not hinder the passage of the vapor jet.

The sanitizing device 1; 100; 1000 comprises at least one dispensing unit 14 housed in a dispensing compartment 16 obtained in a respective sanitizing portion 12; 1020 under the support surface 12′; 1020′.

Each dispensing group 14 comprises at least one dispensing nozzle 18 suitable to direct the vaporized jet towards the surface of the user resting on the support surface, for example the sole of a shoe.

In a preferred embodiment, each dispensing group 14 comprises a plurality of dispensing nozzles 18, distributed under the support surface 12′; 1020′ in such a way that the vapor jets thereby dispensed cover the whole lower surface of the sole of a shoe.

The sanitizing device 1; 100; 1000 further comprises a vaporization group 20 for the vaporization of the sanitizing substance. The vaporization group 20 comprises at least one tank 22; 22a containing the liquid sanitizing substance, one vaporization unit 24 and at least one electric compressor 26.

The vaporization unit 24 is equipped with A vaporization device 25 suitable to cause the sanitizing substance to pass instantaneously from the liquid to the gaseous state in a vaporization chamber 28.

The vaporization unit 24 is fluidically connected to the tank 22 so that the liquid sanitizing substance comes into contact with the vaporization device 25. The vaporization chamber 28 is in fluid communication with each dispensing group 14.

Each electric compressor 26 is fluidically connected to the vaporization unit 24 so as to generate a flow of compressed air which passes through the vaporization chamber 28.

Thus, in the vaporization unit 24 the sanitizing substance passes from the liquid state to the gaseous state. Due to the electric compressor 26, an overpressure is created in the vaporization chamber 28, which is discharged into the atmosphere through the dispensing nozzles 18.

As described hereinafter, the vaporization unit 24 may thus also be positioned at a distance from the dispensing nozzles 28.

In one embodiment, each electric compressor 26 is connected to the vaporization unit 24 via a respective compressed air delivery tube 30. The vaporization unit 24 is connected to each dispensing group 14 by means of a respective vaporized substance delivery tube 32.

For example, as shown in the drawings, each vaporized substance delivery tube 32 ends in a distributor 34 from which secondary tubes 35 branch, carrying the vaporized substance to the dispensing nozzles 18. Each dispensing group 14 therefore comprises a distributor 34, the secondary tubes 35 and the dispensing nozzles 18.

In one embodiment, the mat 10; 110; 1010 is equipped with a presence sensor 36 suitable for detecting the presence of a user on the support surface 12′; 1020′ of at least one sanitizing portion 12; 1020. The vaporization device 25 and at least one electric compressor 26 may be activated by a presence signal generated by the presence sensor 36.

For example, the support surface 12′; 1020′ of at least one sanitizing portion is supported by resilient elements, such as springs, suitable for yielding in the presence of a user on the support surface so as to allow the support surface to be lowered relative to a raised resting position. In this case, the presence sensor 36 may be obtained with a microswitch operated by the support surface 12; 1020′ passing from the raised resting position to the lowered position.

In one embodiment, the mat 10; 100; 1010 comprises at least one passive support portion 40 which forms a passive support surface 40′ of at least one of the user's feet and which is placed next to a respective sanitizing portion 12; 1020.

“Passive portion” means that, unlike the sanitizing portion, it is not adapted to perform an action on the user's footwear when the user simply rests his/her foot on the passive support surface 40′.

In one embodiment, the mat 10; 110; 1010 comprises two sanitizing portions 12; 1020 and two passive support portions 40, arranged alternately therebetween.

Considering, for example, the mat 10; 110; 1010 in the form of a rectangular doormat, the doormat is divided into four substantially equal parts in the lengthwise direction.

In one embodiment, the passive support surface 40′ is equipped with a sole brushing carpet 41, for example placed over a support cover 42.

Therefore, the user may first rest on the passive support surfaces 40′, for example to remove dirt from the soles by rubbing them on the brushing carpets 41′, such as on a traditional doormat. Subsequently, the user may move sideways by placing his/her feet on the sanitizing portions 12; 1020.

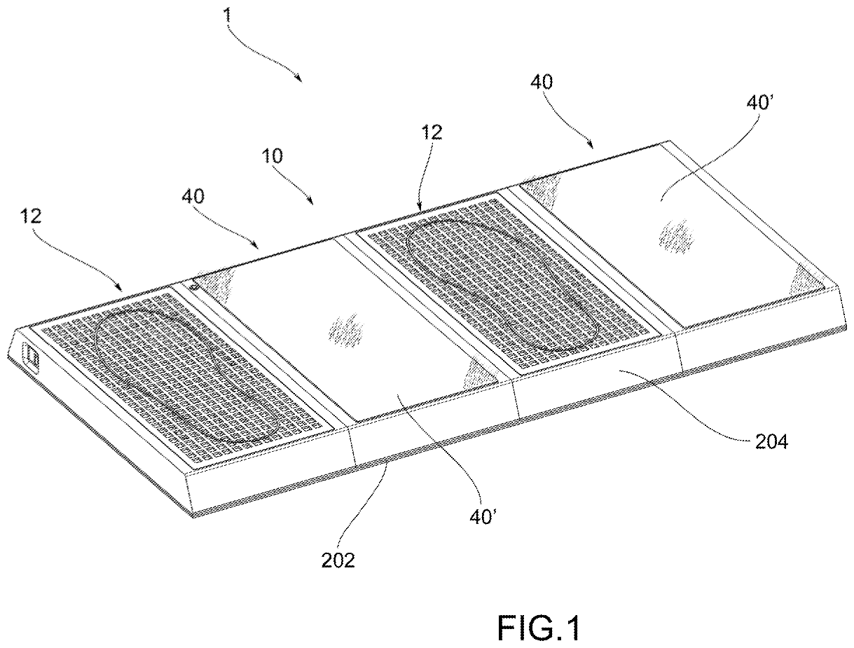

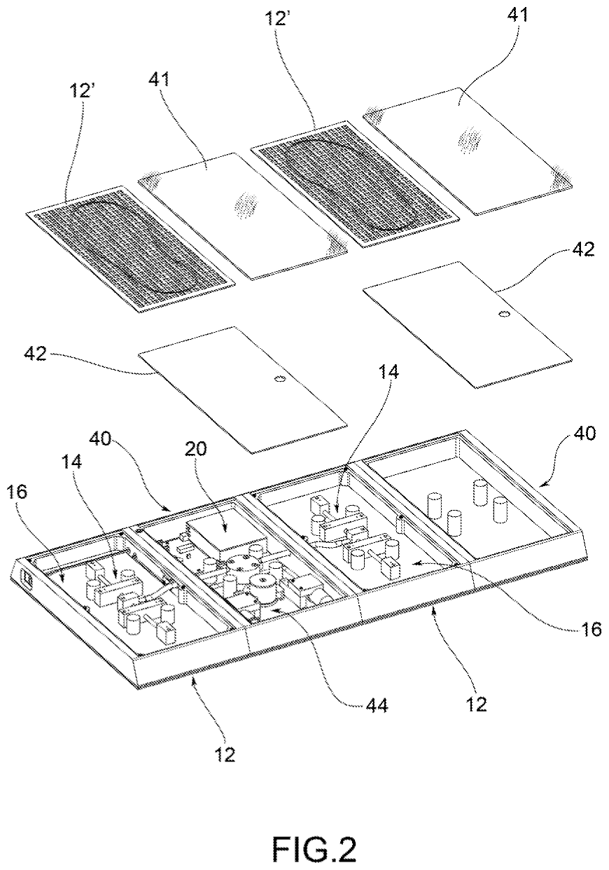

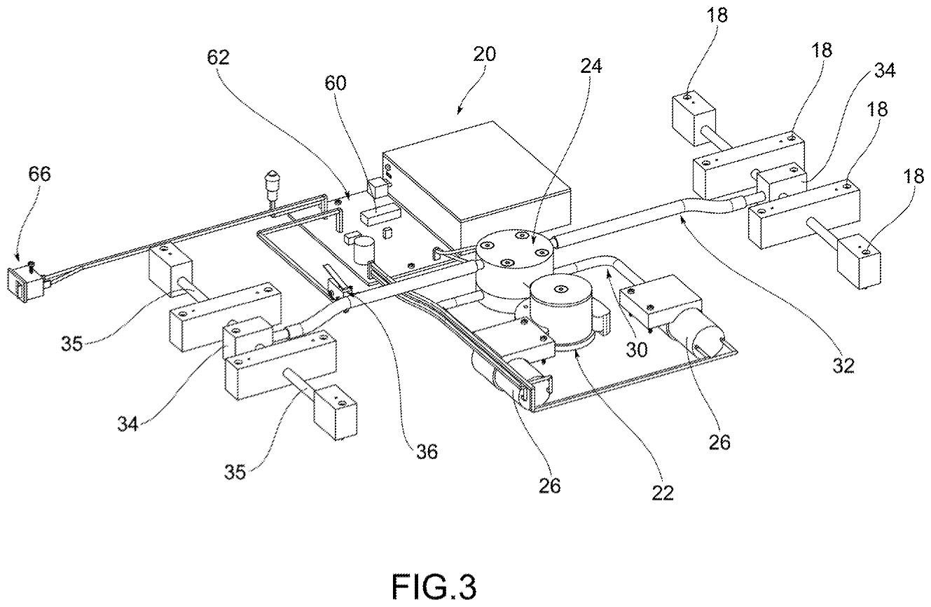

In one embodiment illustrated in FIGS. 1-5, the vaporization group 20 is housed in an inner vaporization compartment 44 obtained in one of the passive support portions 40, for example between the two sanitizing portions 12; 1020, under the passive support surface 40′, comprising, for example, as mentioned above, the cover 42 and the brushing carpet 41.

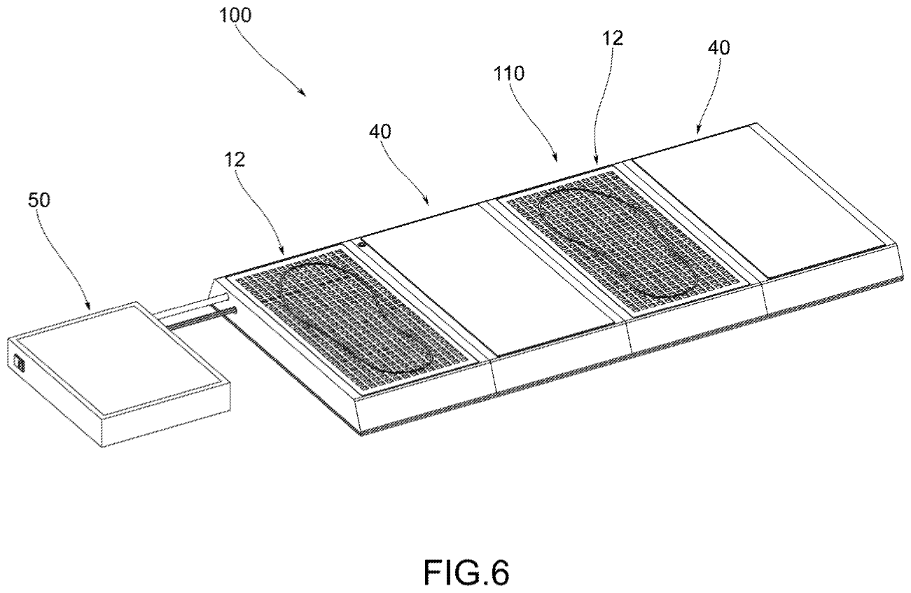

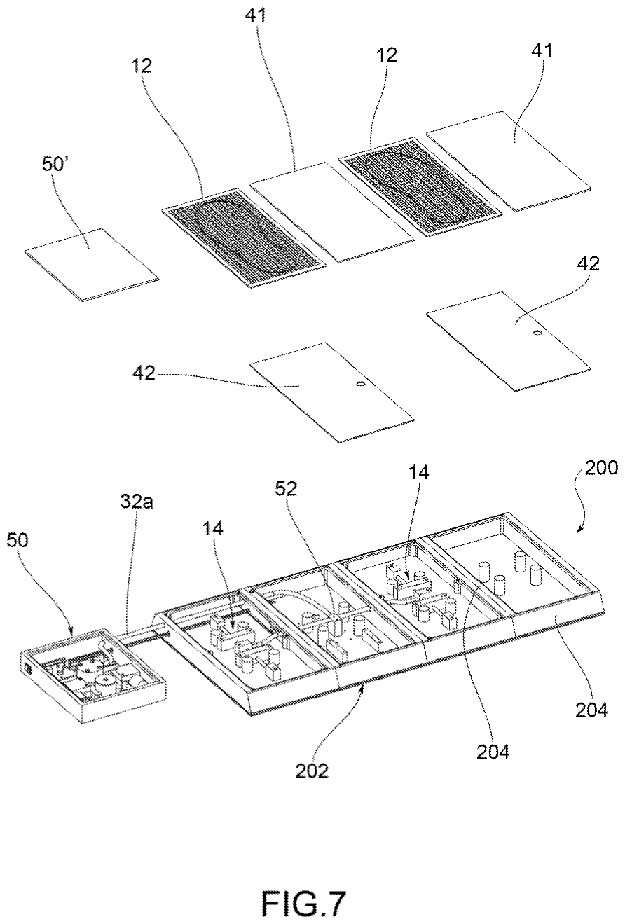

In a variant embodiment shown in FIGS. 6 and 7, the vaporization group 20 is housed in an outer vaporization compartment 50 separate from the mat 100. The outer vaporization compartment 50 may be equipped with a removable cover 50′.

The vaporization group 20 communicates with the dispensing groups 14 by means of at least one vaporized substance delivery tube 32a connecting the vaporization unit 24 housed in the vaporization compartment 50 to the mat 100.

For example, the vaporized substance delivery tube 32a ends in an inlet connection element 52 housed in the mat 100 and connected in turn to the dispensing groups 14 in the mat 100.

This embodiment is particularly suitable, for example, for industrial applications of the sanitizing device in particularly harsh environments, which require the safe management of electronic devices, in this case the components of the vaporization group 20.

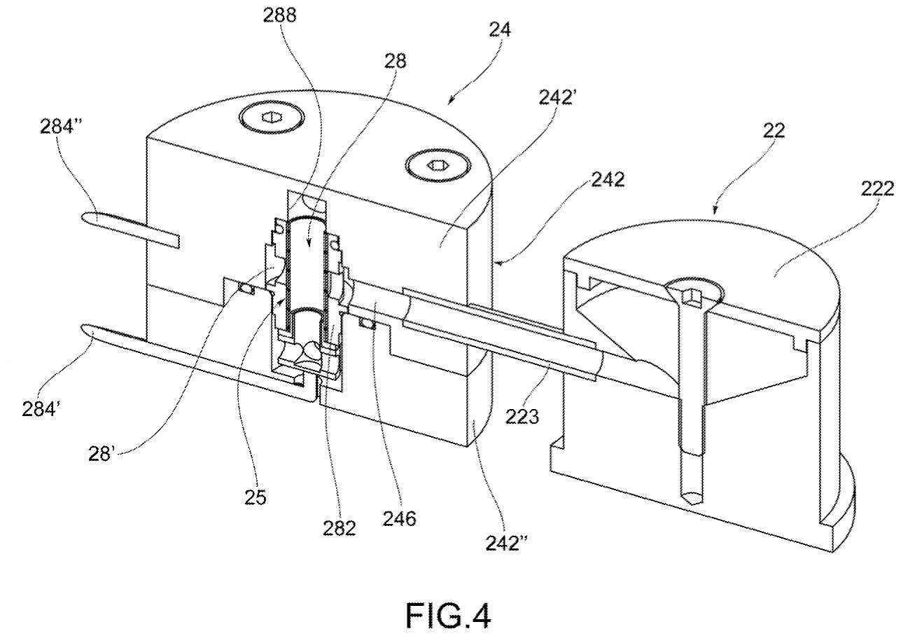

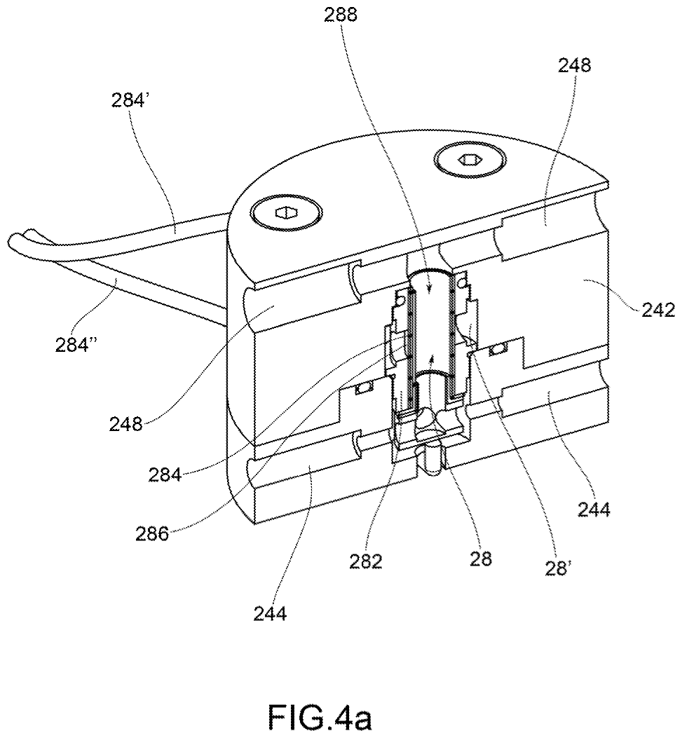

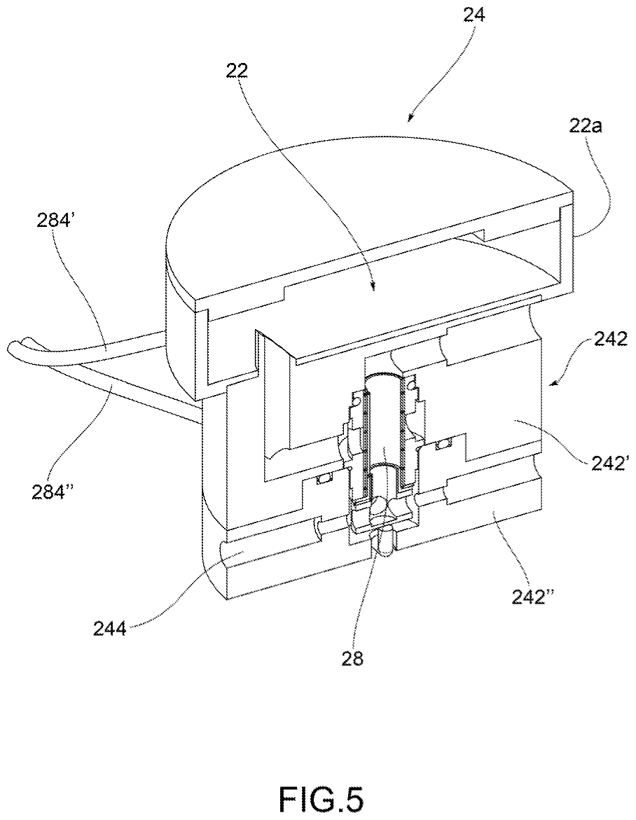

In one embodiment (FIGS. 4, 4a, 5), the vaporization unit 24 comprises a unit body 242 wherein are formed the vaporization chamber 28, at least one air inlet passage 244 fluidically connectable to an electric compressor 26 and flowing into the vaporization chamber 28, at least one substance inlet passage 246 fluidically connectable to the tank 22 of the sanitizing substance and flowing into the vaporization chamber 28, and at least one outlet passage 248 extending from the vaporization chamber 28 and which is fluidically connectable to a respective dispensing group 14.

A vaporization insert 282 is sealingly housed in the vaporization chamber 28. This vaporization insert 282 supports an electrical resistor 284, e.g. with a helical shape, which may be activated for vaporization of the sanitizing substance. In one embodiment, the electrical resistor 284 is embedded in a protective jacket 286 made of a porous material, i.e. permeable to the liquid sanitizing substance coming from the tank 22, such as fabric.

More specifically, the vaporization insert 282, and thus the electrical resistor 284, is penetrated axially by an insert through-hole 288 the opposing ends of which are in fluid communication with the air inlet passage 244 and the outlet passage 248, respectively. Moreover, the substance inlet passage 246 flows into an annular portion 28′ of the vaporization chamber 28 surrounding an intermediate portion of the electrical resistor 284.

In one embodiment, such annular chamber 28′ extends substantially coaxially to the through-hole 288. For example, the axial insert hole 288 extends axially in the unit body 242; the sanitizing substance inlet passage 246 extends, at least in one of the end portions thereof, radially in the unit body 242.

The electrical resistor 284 is powered by two power cables 284′, 284″. In one embodiment, the unit body 242 is formed by the coupling of a first portion 242′, for example, upper, made of an electroconductive material, such as a metallic material, with a second portion 242″, for example, lower, made of an insulating material, for example, a polymeric material. The first portion 242′ is in contact with the electrical resistor 284 and may therefore act as an electrical contact connecting a power cable 284″ to the electrical resistor 284.

In one embodiment, the liquid sanitizing substance contained in the tank 22 is fed by gravity to the vaporization device. In this way, the liquid sanitizing substance is always in contact with the vaporization device. As soon as these are activated, for example by the presence signal of the user, there is thus the instantaneous vaporization of the sanitizing substance present in the annular portion 28′ of the vaporization chamber 28.

In one embodiment shown in FIGS. 4 and 4a, the tank 22 is obtained in a tank body 222 separate from the vaporization unit 24 and connected to the latter by means of a tank tube 223.

In a variant embodiment shown in FIG. 5, the tank is instead obtained in a head portion 222a of the vaporization unit 24.

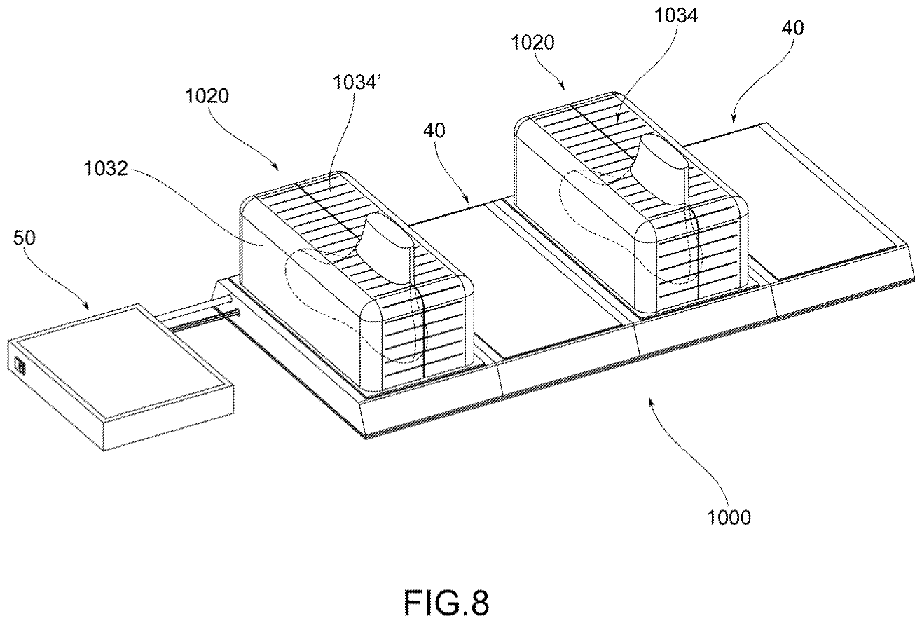

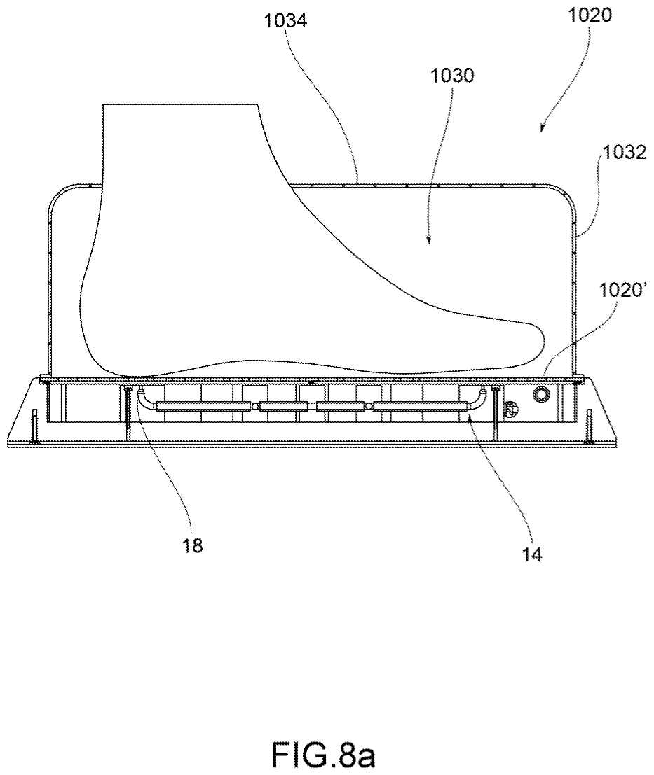

In a further variant embodiment 1000 of the sanitizing device, illustrated in FIGS. 8 and 8a, the support surface 1020′ of each sanitizing portion 1020 forms the bottom wall of a closed sanitizing compartment 1030, delimited by side walls 1032 and a top wall 1034 and suitable for accomodating the foot and ankle of a user.

In one embodiment, at least the top wall 1034 is formed from flexible elements 1034′ suitable to allow the insertion of the foot in the sanitizing compartment 1030. When the user's foot is inserted in the sanitizing compartment 1030, the flexible elements 1034′ adhere to the user's limb exerting a substantial sealing action that prevents the steam jet of the sanitizing substance from leaving the sanitizing compartment.

This embodiment is particularly suitable for professional applications, where, for example, the sanitization is required not only for the sole of the shoe, but also for the side and upper parts of the same shoe. To this end, dispensing nozzles 18 may also be provided in peripheral positions of the support surface 1020′ and thus outside the support area of the sole.

In one embodiment, the electrical/electronic devices of the vaporization group 20 are controlled by an electronic control unit 60, e.g. mounted on a printed circuit board 62 housed in the vaporization compartment 44; 50. The circuit board 62 may be powered by a battery pack 64, e.g. also housed in the vaporization compartment 44; 50, and/or by the mains voltage via a power cable.

The sanitizing device may also be equipped with a main switch 66 that enables or disables the power supply to the circuit board 62.

In one embodiment, the control unit 60 also comprises a communication interface for connecting to a remote-control device, for example a smartphone or a home automation system, so as to allow the operating status of the device to be controlled remotely. For example, the liquid level in the tank 22, the presence sensor, the battery charge level, the sanitation cycle time, etc. may be set and/or monitored.

In one embodiment, the mat 10; 110; 1010 comprises a frame 200 that forms the support base 202 for the sole and side walls 204 that delimit the compartments for the sanitization and dispensing groups.

To the embodiments of the sanitizing device according to the disclosure, one skilled in the art, to satisfy contingent needs, may make modifications, adaptations and replacements of some elements with others that are functionally equivalent, without departing from the scope of the following claims. Each of the features described as belonging to a possible embodiment may be implemented independently from the other described embodiments.

1. A sanitizing device, comprising a treadable mat wherein is formed at least one sanitizing portion forming a support surface which may be walked on by a user and which is suitable for dispensing a liquid sanitizing substance in vaporized jet form, the sanitizing device further comprising:
at least one dispensing group housed in a dispensing compartment formed in a respective sanitizing portion below said support surface and comprising at least one dispensing nozzle suitable to direct the vaporized jet towards a surface of the user resting on the support surface, including a sole of a shoe;
a vaporization group comprising:
at least one tank containing the liquid sanitizing substance,
a vaporization unit comprising a vaporization device suitable to cause an instantaneous passage of the liquid sanitizing substance from a liquid state to a gaseous state in a vaporization chamber, the vaporization unit being fluidically connected to the at least one tank so that the liquid sanitizing substance is in contact with the vaporization device, the vaporization chamber being in fluid communication with the at least one dispensing group;
at least one electric compressor fluidically connected to the vaporization unit to generate a flow of compressed air that passes through the vaporization chamber.

2. The sanitizing device of claim 1, wherein the treadable mat is comprises a presence sensor detecting presence of the user on the support surface of at least one sanitizing portion, the vaporization device and the at least one electric compressor being activated by a presence signal generated by said presence sensor.

3. The sanitizing device of claim 1, wherein the treadable mat comprises at least one passive support portion forming a passive support surface of at least one of the user's feet and which is placed next to a respective sanitizing portion.

4. The sanitizing device of claim 3, wherein the vaporization group is housed in an inner vaporization compartment formed in said at least one passive support portion, below the passive support surface.

5. The sanitizing device of claim 1, wherein the vaporization group is housed in a vaporization compartment outside the treadable mat and communicates with the at least one dispensing group through at least one vaporized substance delivery tube connecting the vaporization unit housed in the vaporization compartment to the treadable mat.

6. The sanitizing device of claim 1, wherein the vaporization unit further comprises:
a unit body wherein are formed the vaporization chamber, at least one air inlet passage fluidically connectable to the at least one electric compressor and flowing into the vaporization chamber, at least one substance inlet passage fluidically connectable to the at least one tank containing the liquid sanitizing substance and flowing into the vaporization chamber, and at least one outlet passage extending from the vaporization chamber and fluidically connectable to the at least one dispensing group;
a vaporization insert sealingly housed in the vaporization chamber and bearing an electrical resistor which may be activated for vaporization of the liquid sanitizing substance, said electrical resistor being embedded in a protective jacket made of a porous material.

7. The sanitizing device of claim 6, wherein the vaporization insert is axially penetrated by an insert through hole of which opposite ends are in fluid communication with the at least one air inlet passage and with the at least one outlet passage, respectively, and wherein the at least one substance inlet passage flows into an annular portion of the vaporization chamber which surrounds an intermediate portion of the electrical resistor, an annular chamber being coaxial to the through hole.

8. The sanitizing device of claim 1, wherein the liquid sanitizing substance contained in the at least one tank is fed by gravity to the vaporization device.

9. The sanitizing device of claim 2, wherein the support surface of at least one sanitizing portion is supported by resilient elements suitable to yield in the presence of the user on the support surface to allow a lowering of the support surface with respect to a raised rest position, the presence sensor being constructed with a microswitch operable by the support surface passing into a lowered position.

10. The sanitizing device of claim 1, wherein the support surface of each sanitizing portion forms a bottom wall of a closed sanitizing compartment, delimited by side walls and a top wall and suitable to receive the user's foot and ankle.

11. The sanitizing device of claim 10, wherein at least the top wall is formed by flexible elements allowing insertion of the user's foot into the sanitizing compartment.

12. The sanitizing device of claim 1, wherein the treadable mat is formed by two sanitizing portions and by two passive support portions, arranged alternately with each other, the passive support surface being provided with a shoe sole brushing carpet.

13. The sanitizing device of claim 1, wherein the vaporization device is governed by an electronic control unit powered by a battery pack housed inside the treadable mat or by mains voltage through a power supply cable.

14. The sanitizing device of claim 1, further comprising a communication interface for connecting to a remote-control device, including a smartphone or a home automation system, to allow an operating status of the sanitizing device to be controlled remotely.
